(19) United States
(12) Patent Application Publication
(10) Pub. No.: US 2026/0272906 A1
(43) Pub. Date: Sep. 17, 2026

(54) PERIPHERALLY RESTRICTED LPAR1 ANTAGONISTS AND USES THEREOF

(71) Applicant: CONTINEUM THERAPEUTICS, INC., San Diego, CA (US)

(72) Inventor: Austin Chih-Yu CHEN, San Diego, CA (US)

(21) Appl. No.: 19/420,156

(22) Filed: Dec. 15, 2025

Related U.S. Application Data
(63) Continuation of application No. PCT/US25/19389, filed on Mar. 11, 2025.
(60) Provisional application No. 63/564,423, filed on Mar. 12, 2024.

(51) Int. Cl. A61K 31/444; C07D 213/75

(52) U.S. Cl. CPC A61K 31/444; C07D 213/75

(57) ABSTRACT

Described herein, inter alia, are peripherally restricted LPAR1 antagonists and uses thereof.

### CROSS-REFERENCE TO RELATED APPLICATIONS

This application claims the benefit of U.S. Provisional Application No. 63/564,423 filed Mar. 12, 2024, which is incorporated herein by reference in its entirety and for all purposes.

### BACKGROUND

Lysophosphatidic acid, or LPA, is a family of bioactive phospholipids that are associated with multiple cellular functions. While the family members differ with regards to the length and the degree of saturation of their respective long-chain fatty acid backbone (Fujiwara et al., J. Biol. Chem., 2005, 280, 35038-35050), they are all capped by a glycerol-phosphate group through an ester linkage. LPAs are produced biologically from membrane phospholipids through a multi-step cascade mediated by enzymes that include lysophospholipase D (lysoPLD), autotaxin (ATX), phospholipase A1 (PLA1), phospholipase A2 (PLA2) and acylglycerol kinase (AGK) (Mutoh et al., British J. Pharmacol., 2012, 165, 829-844). Once formed, the LPAs can regulate numerous cellular signaling pathways by binding to a class of 7-membrane domain G protein-coupled receptors (GPCRs), collectively known as LPA receptors (LPARs), of which six have been characterized: LPAR1, LPAR2, LPAR3, LPAR4, LPAR5, and LPAR6 (Choi, J. W., Annu. Rev. Pharmacol. Toxicol., 2010, 50, 157-186). The biological responses elicited by the binding of LPAs to LPARs are both wide-ranging and context-dependent (Yung et al., J. Lipid Res. 2014, 55, 1192-1214; Yung et al., Neuron 2015, 85, 669-682). These can include induction of cell proliferation, stimulation of cell migration and contraction, promotion of neurite retraction, suppression of apoptosis, initiation of chemotaxis, closure of gap junction, and others (Chun et al., Editors, Lysophospholipid Receptors: Signaling and Biochemistry, 2013, Wiley, ISBN: 978-0-470-56905-4). Furthermore, aberrant upregulation of the LPA pathway has been implicated in multiple diseases, including cancer, inflammatory diseases, infertility, neuropathic pain, psychotic and neurodegenerative disorders, atherosclerosis, as well as fibrosis of the skin, kidney, lung, and liver (Choi, J. W., Annu. Rev. Pharmacol. Toxicol., 2010, 50, 157-186; Noguchi et al., Curr. Opin Pharmacol., 2009, 9, 15-23; Yanagida et al., J. Biochem., 2011, 150, 223-232). Consequently, the targeting of LPA receptors has been, and continues to be an area of intense interest for the identification of potential treatments for these disorders. Disclosed herein, inter alia, are solutions to these and other problems in the art.

### BRIEF SUMMARY

In an aspect is provided a compound, or a pharmaceutically acceptable salt or solvate thereof, having the formula:

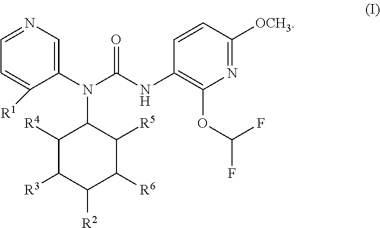

R¹ is isopropyl or isobutyl.

R²
is —C(O)OH, —CH₂C(O)OH, —CH₂CH₂C(O)OH, —C(O)NHS(O)₂R^{2A}, —P(O)(OH)₂, —P(O)(OH)R^{2A}, —SO₃H, —SO₂H, —NHC(O)NHS(O)₂R^{2A},

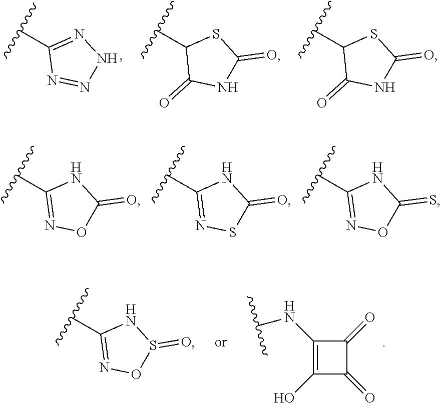

R^{2A} is unsubstituted C₁-C₄ alkyl.

R³, R⁴, R⁵, and R⁶ are independently hydrogen or unsubstituted C₁-C₄ alkyl.

In an aspect is provided a compound, or a pharmaceutically acceptable salt or solvate thereof, having the formula:

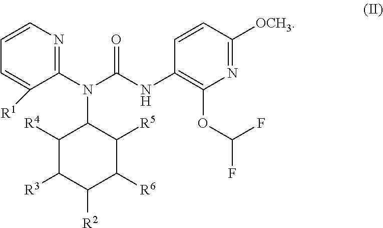

R¹, R², R³, R⁴, R⁵, and R⁶ are as described herein, including in embodiments.

In an aspect is provided a pharmaceutical composition including a compound described herein, or a pharmaceutically acceptable salt or solvate thereof, and a pharmaceutically acceptable excipient.

In an aspect is provided a method of treating a peripherally restricted LPAR1-associated disease in a subject in need thereof, the method including administering to the subject a therapeutically effective amount of a compound described herein, or a pharmaceutically acceptable salt of solvate thereof.

In an aspect is provided a method of modulating LPAR1 activity in a subject, the method including administering to the subject a compound described herein, or a pharmaceutically acceptable salt or solvate thereof.

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di-, and multivalent radicals. The alkyl may include a designated number of carbons (e.g., C₁-C₁₀ means one to ten carbons). In embodiments, the alkyl is fully saturated. In embodiments, the alkyl is monounsaturated. In embodiments, the alkyl is polyunsaturated. Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkenyl includes one or more double bonds. An alkynyl includes one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. The term “alkynylene” by itself or as part of another substituent, means, unless otherwise stated, a divalent radical derived from an alkyne. In embodiments, the alkylene is fully saturated. In embodiments, the alkylene is monounsaturated. In embodiments, the alkylene is polyunsaturated. An alkenylene includes one or more double bonds. An alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —S—CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′- and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from a heteroalkyne. In embodiments, the heteroalkylene is fully saturated. In embodiments, the heteroalkylene is monounsaturated. In embodiments, the heteroalkylene is polyunsaturated. A heteroalkenylene includes one or more double bonds. A heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. A bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. A bicyclic or multicyclic cycloalkenyl ring system refers to multiple rings fused together wherein at least one of the fused rings is a cycloalkenyl ring and wherein the multiple rings are attached to the parent molecular moiety through any carbon atom contained within a cycloalkenyl ring of the multiple rings.

In embodiments, the term “heterocycloalkyl” means a monocyclic, bicyclic, or a multicyclic heterocycloalkyl ring system. In embodiments, heterocycloalkyl groups are fully saturated. A bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g., all rings being substituted heterocycloalkylene wherein each ring may be the same or different substituted heterocycloalkylene). When referring to a spirocyclic ring system, heterocyclic spirocyclic rings means spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

Bridged rings are two ore more rings that share three or more atoms, separating the two bridgehead atoms by a bridge containing at least one atom. Individual rings in bridged rings may be substituted or unsubstituted and may have different substituents from other individual rings within a set of bridged rings. Possible substituents for individual rings within bridged rings are the possible substituents for the same ring when not part of bridged rings (e.g., substituents for cycloalkyl or heterocycloalkyl rings). Bridged rings may be substituted or unsubstituted cycloalkyl, substituted or unsubstituted cycloalkylene, substituted or unsubstituted heterocycloalkyl or substituted or unsubstituted heterocycloalkylene and individual rings within a bridged ring group may be any of the immediately previous list, including having all rings of one type (e.g., all rings being substituted heterocycloalkylene wherein each ring may be the same or different substituted heterocycloalkylene). When referring to a bridged ring system, heterocyclic bridged rings means bridged rings wherein at least one ring is a heterocyclic ring and wherein each ring may be a different ring. When referring to a bridged ring system, substituted bridged rings means that at least one ring is substituted and each substituent may optionally be different.

The symbol “~” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

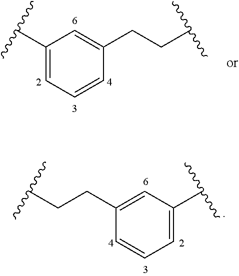 [SMILES: *CCc1cccc(*)c1 ; *CCc1cccc(*)c1]

An alkylarylene moiety may be substituted (e.g., with a substituent group) on the alkylene moiety or the arylene linker (e.g., at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂ CH₃, —SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, =O, =NR′, ═N—OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′ C(O)NR″R′″, —NR″C(O)₂R′, —NRC(NR′R″R′″)═NR″″, —NRC(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups.

When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′ C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O) NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), selenium (Se), and silicon (Si). In embodiments, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NI—₂, —NIHINH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
   (i) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NI—₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
      (a) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NI—₂, —NHNI₂, —ONH₂, —NHC(O)NHNII₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and each substituted or unsubstituted heteroaryl is a substituted or unsubstituted 5 to 6 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

“Analog,” “analogue,” or “derivative” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an”, as used in herein means one or more. In addition, the phrase “substituted with a[n]”, as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl”, the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13.A}, R^{13.B}, R^{13.C}, R^{13.D}, etc., wherein each of R^{13.A}, R^{13.B}, R^{13.C}, R^{13.D}, etc. is defined within the scope of the definition of R¹³ and optionally differently. Where an R moiety, group, or substituent as disclosed herein is attached through the representation of a single bond and the R moiety, group, or substituent is oxo, a person having ordinary skill in the art will immediately recognize that the oxo is attached through a double bond in accordance with the normal rules of chemical valency.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

A polypeptide, or a cell is “recombinant” when it is artificial or engineered, or derived from or contains an artificial or engineered protein or nucleic acid (e.g., non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, the certain methods presented herein successfully treat cancer by decreasing the incidence of cancer and or causing remission of cancer. In some embodiments of the compositions or methods described herein, treating cancer includes slowing the rate of growth or spread of cancer cells, reducing metastasis, or reducing the growth of metastatic tumors. The term “treating” and conjugations thereof, include prevention of an injury, pathology, condition, or disease. In embodiments, treating is preventing. In embodiments, treating does not include preventing. In embodiments, the treating or treatment is not prophylactic treatment.

An “effective amount” is an amount sufficient for a compound to accomplish a stated purpose relative to the absence of the compound (e.g., achieve the effect for which it is administered, treat a disease, reduce enzyme activity, increase enzyme activity, reduce signaling pathway, reduce one or more symptoms of a disease or condition. An example of an “effective amount” is an amount sufficient to contribute to the treatment, prevention, or reduction of a symptom or symptoms of a disease, which could also be referred to as a “therapeutically effective amount” when referred to in this context. A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. An “activity increasing amount,” as used herein, refers to an amount of agonist required to increase the activity of an enzyme relative to the absence of the agonist. A “function increasing amount,” as used herein, refers to the amount of agonist required to increase the function of an enzyme or protein relative to the absence of the agonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity (e.g., signaling pathway) of a protein in the absence of a compound as described herein (including embodiments, examples, figures, or Tables).

“Contacting” is used in accordance with its plain ordinary meaning and refers to the process of allowing at least two distinct species (e.g., chemical compounds including biomolecules, or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be a compound as described herein and a cellular component (e.g., protein, ion, lipid, nucleic acid, nucleotide, amino acid, protein, particle, organelle, cellular compartment, microorganism, virus, lipid droplet, vesicle, small molecule, protein complex, protein aggregate, or macromolecule). In some embodiments contacting includes allowing a compound described herein to interact with a cellular component (e.g., protein, ion, lipid, nucleic acid, nucleotide, amino acid, protein, particle, virus, lipid droplet, organelle, cellular compartment, microorganism, vesicle, small molecule, protein complex, protein aggregate, or macromolecule) that is involved in a signaling pathway.

As defined herein, the term “activation,” “activate,” “activating” and the like in reference to a protein refers to conversion of a protein into a biologically active derivative from an initial inactive or deactivated state. The terms reference activation, or activating, sensitizing, or up-regulating signal transduction or enzymatic activity or the amount of a protein decreased in a disease.

The terms “agonist,” “activator,” “upregulator,” etc. refer to a substance capable of detectably increasing the expression or activity of a given gene or protein. The agonist can increase expression or activity by at least 10%, 20%, 30%, 40%, 50%, 60%, 70%, 80%, 90%, 95%, 96%, 97%, 98%, or 99% in comparison to a control in the absence of the agonist. In certain instances, expression or activity is 1.5-fold, 2-fold, 3-fold, 4-fold, 5-fold, 10-fold or higher than the expression or activity in the absence of the agonist.

As defined herein, the term “inhibition,” “inhibit,” “inhibiting” and the like in reference to a cellular component-inhibitor interaction means negatively affecting (e.g., decreasing) the activity or function of the cellular component (e.g., decreasing the signaling pathway stimulated by a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)), relative to the activity or function of the cellular component in the absence of the inhibitor. In embodiments inhibition means negatively affecting (e.g., decreasing) the concentration or levels of the cellular component relative to the concentration or level of the cellular component in the absence of the inhibitor. In some embodiments, inhibition refers to reduction of a disease or symptoms of disease. In some embodiments, inhibition refers to a reduction in the activity of a signal transduction pathway or signaling pathway (e.g., reduction of a pathway involving the cellular component). Thus, inhibition includes, at least in part, partially or totally blocking stimulation, decreasing, preventing, or delaying activation, or inactivating, desensitizing, or down-regulating the signaling pathway or enzymatic activity or the amount of a cellular component.

The terms “inhibitor,” “repressor,” “antagonist,” or “downregulator” interchangeably refer to a substance capable of detectably decreasing the expression or activity of a given gene or protein. The antagonist can decrease expression or activity by at least 10%, 20%, 30%, 40%, 50%, 60%, 70%, 80%, 90%, 95%, 96%, 97%, 98%, or 99% in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “lysophosphatidic acid receptor 1 antagonist” or “LPAR1 antagonist” refers to any exogenously administered compound or agent that is capable of partially or completely inhibiting, or reversing, the effect of an agonist (e.g., lysophosphatidic acid) on the LPAR1 receptor. The term is inclusive of compounds or agents characterized or described as antagonists, partial antagonists, and negative allosteric modulators.

The term “modulator” refers to a composition that increases or decreases the level of a target molecule or the function of a target molecule or the physical state of the target of the molecule (e.g., a target may be a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)) relative to the absence of the composition.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

“Patient”, “patient in need thereof”, “subject”, or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In embodiments, a patient is human. In embodiments, a patient in need thereof is human. In embodiments, a subject is human. In embodiments, a subject in need thereof is human.

“Disease” or “condition” refer to a state of being or health status of a patient or subject capable of being treated with the compounds or methods provided herein. In some embodiments, the disease is a disease related to (e.g., caused by) a cellular component (e.g., protein, ion, lipid, nucleic acid, nucleotide, amino acid, protein, particle, organelle, cellular compartment, microorganism, vesicle, small molecule, protein complex, protein aggregate, or macromolecule). In embodiments, the disease is a fibrotic disease. In embodiments, the disease is idiopathic pulmonary fibrosis. In embodiments, the disease is renal fibrosis. In embodiments, the disease is nonalcoholic steatohepatitis. In embodiments, the disease is scleroderma. In embodiments, the disease is a cancer. In embodiments, the disease is ovarian cancer. In embodiments, the disease is breast cancer. In embodiments, the disease is liver cancer. In embodiments, the disease is gastric cancer. In embodiments, the disease is pancreatic cancer. In embodiments, the disease is lung cancer. In embodiments, the disease is osteosarcoma.

As used herein, the terms “fibrotic disease” and “fibrosis” refer to any disease or condition characterized by the formation of excess fibrous connective tissue. The formation of excess fibrous connective tissue may be in response to a reparative or reactive process. Fibrotic diseases include but are not limited to pulmonary fibrosis (e.g., idiopathic pulmonary fibrosis (IPF)), liver fibrosis (e.g., nonalcoholic steatohepatitis (NASH)), myelofibrosis, skin fibrosis (e.g., scleroderma), ocular fibrosis, mediastinal fibrosis, cardiac fibrosis, kidney fibrosis, stromal fibrosis, epidural fibrosis, epithelial fibrosis, or idiopathic fibrosis.

As used herein, the term “cancer” refers to all types of cancer, neoplasm or malignant tumors found in mammals (e.g., humans), including leukemia, lymphoma, carcinomas and sarcomas. Exemplary cancers that may be treated with a compound or method provided herein include cancer of the thyroid, endocrine system, breast, cervix, colon, head and neck, liver, kidney, lung, non-small cell lung, melanoma, mesothelioma, ovary, sarcoma, stomach, uterus, colorectal cancer, or pancreatic cancer. Additional examples include, Hodgkin's Disease, Non-Hodgkin's Lymphoma, multiple myeloma, rhabdomyosarcoma, primary thrombocytosis, primary macroglobulinemia, malignant pancreatic insulanoma, malignant carcinoid, urinary bladder cancer, premalignant skin lesions, testicular cancer, lymphomas, thyroid cancer, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cutaneous T-cell lymphoma, peripheral T-cell lymphoma, anaplastic large cell lymphoma, mycosis fungoides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the term “peripherally restricted LPAR1-associated disease” refers to any disease or condition caused by aberrant activity or signaling of LPAR1 outside of the central nervous system. For example, the following diseases would be considered to be caused by aberrant activity or signalling inside the central nervous system and would therefore not be included within the term “peripherally restricted LPAR1-associated disease”: Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, frontotemporal dementia, Gerstmann-Straussler-Scheinker syndrome, Huntington's disease, HIV-associated dementia, Kennedy's disease, Krabbe's disease, kuru, Lewy body dementia, Machado-Joseph disease (Spinocerebellar ataxia type 3), Multiple sclerosis, Multiple System Atrophy, Narcolepsy, Neuroborreliosis, Parkinson's disease, Pelizaeus-Merzbacher Disease, Pick's disease, Primary lateral sclerosis, Prion diseases, Refsum's disease, Sandhoff's disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, Tabes dorsalis, brain cancer, medulloblastoma, neuroblastoma, glioma, glioblastoma multiforme, primary brain tumors, astrocytomas, atypical teratoid/rhabdoid tumor, craniopharyngioma, ependymoblastoma, ependymoma, retinoblastoma, medulloepithelioma, pineal parenchymal tumors of intermediate differentiation, pienoblastoma and supratentorial primitive neuroectodermal tumors, pituitary tumor, and primary central nervous system lymphoma. In embodiments, the peripherally restricted LPAR1-associated disease is a fibrotic disease. In embodiments, the peripherally restricted LPAR1-associated disease is idiopathic pulmonary fibrosis. In embodiments, the peripherally restricted LPAR1-associated disease is renal fibrosis. In embodiments, the peripherally restricted LPAR1-associated disease is nonalcoholic steatohepatitis. In embodiments, the peripherally restricted LPAR1-associated disease is scleroderma. In embodiments, the peripherally restricted LPAR1-associated disease is a cancer. In embodiments, the peripherally restricted LPAR1-associated disease is ovarian cancer. In embodiments, the peripherally restricted LPAR1-associated disease is breast cancer. In embodiments, the peripherally restricted LPAR1-associated disease is liver cancer. In embodiments, the peripherally restricted LPAR1-associated disease is gastric cancer. In embodiments, the peripherally restricted LPAR1-associated disease is pancreatic cancer. In embodiments, the peripherally restricted LPAR1-associated disease is lung cancer. In embodiments, the peripherally restricted LPAR1-associated disease is osteosarcoma.

The term “drug” is used in accordance with its common meaning and refers to a substance which has a physiological effect (e.g., beneficial effect, is useful for treating a subject) when introduced into or to a subject (e.g., in or on the body of a subject or patient). A drug moiety is a radical of a drug.

A “detectable agent,” “detectable compound,” “detectable label,” or “detectable moiety” is a substance (e.g., element), molecule, or composition detectable by spectroscopic, photochemical, biochemical, immunochemical, chemical, magnetic resonance imaging, or other physical means. For example, detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, 99Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g., fluorescent dyes), modified oligonucleotides (e.g., moieties described in PCT/US2015/022063, which is incorporated herein by reference), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g., carbon-11, nitrogen-13, oxygen-15, fluorine-18, rubidium-82), fluorodeoxyglucose (e.g., fluorine-18 labeled), any gamma ray emitting radionuclides, positron-emitting radionuclide, radiolabeled glucose, radiolabeled water, radiolabeled ammonia, biocolloids, microbubbles (e.g., including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g., iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb, and Lu.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

As used herein, the term “administering” is used in accordance with its plain and ordinary meaning and includes oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

The compounds described herein can be used in combination with one another, with other active agents known to be useful in treating a disease associated with cells expressing a disease associated cellular component, or with adjunctive agents that may not be effective alone, but may contribute to the efficacy of the active agent.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

In therapeutic use for the treatment of a disease, compound utilized in the pharmaceutical compositions of the present invention may be administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound or drug being employed. For example, dosages can be empirically determined considering the type and stage of disease (e.g., fibrotic disease or cancer) diagnosed in a particular patient. The dose administered to a patient, in the context of the present invention, should be sufficient to affect a beneficial therapeutic response in the patient over time. The size of the dose will also be determined by the existence, nature, and extent of any adverse side effects that accompany the administration of a compound in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

The term “associated” or “associated with” in the context of a substance or substance activity or function associated with a disease (e.g., a protein associated disease, disease associated with a cellular component) means that the disease (e.g., fibrotic disease or cancer) is caused by (in whole or in part), or a symptom of the disease is caused by (in whole or in part) the substance or substance activity or function or the disease or a symptom of the disease may be treated by modulating (e.g., inhibiting or activating) the substance (e.g., cellular component). As used herein, what is described as being associated with a disease, if a causative agent, could be a target for treatment of the disease.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g., by administering a compound or using a method as described herein), results in reduction of the disease or one or more disease symptoms.

The term “isolated,” when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide,” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

The term “protein complex” is used in accordance with its plain ordinary meaning and refers to a protein which is associated with an additional substance (e.g., another protein, protein subunit, or a compound). Protein complexes typically have defined quaternary structure. The association between the protein and the additional substance may be a covalent bond. In embodiments, the association between the protein and the additional substance (e.g., compound) is via non-covalent interactions. In embodiments, a protein complex refers to a group of two or more polypeptide chains. Proteins in a protein complex are linked by non-covalent protein-protein interactions. A non-limiting example of a protein complex is the proteasome.

The term “lysophosphatidic acid receptor” or “LPAR” refers to one or more of the family of G protein-coupled receptors for lysophosphatidic acid (LPA). In embodiments, LPAR includes LPAR1, LPAR2, LPAR3, LPAR4, LPAR5, and LPAR6.

The term “lysophosphatidic acid receptor 1” or “LPAR1” refers to a G protein-coupled receptor (including homologs, isoforms, and functional fragments thereof) that binds the lipid signaling molecule lysophosphatidic acid (LPA). The term includes any recombinant or naturally-occurring form of LPAR1 variants thereof that maintain LPAR1 activity (e.g., within at least 30%, 40%, 50%, 60%, 70%, 80%, 90%, 95%, or 100% activity compared to wildtype LPAR1). In embodiments, the LPAR1 protein encoded by the LPAR1 gene has the amino acid sequence set forth in or corresponding to Entrez 1902, UniProt Q92633, RefSeq (protein) NP_001392.2 or RefSeq (protein) NP_476500.1. In embodiments, the LPAR1 gene has the nucleic acid sequence set forth in RefSeq (mRNA) NM_001401.3 or RefSeq (mRNA) NM_057159.2. In embodiments, the amino acid sequence or nucleic acid sequence is the sequence known at the time of filing of the present application.

The term “selective” or “selectivity” or the like in reference to a compound or agent refers to the compound's or agent's ability to cause an increase or decrease in activity of a particular molecular target (e.g., protein, enzyme, etc.) preferentially over one or more different molecular targets (e.g., a compound having selectivity toward lysophosphatidic acid receptor 1 (LPAR1) would preferentially inhibit LPAR1 over other lysophosphatidic acid receptors). In embodiments, an “lysophosphatidic acid receptor 1 selective compound” or “LPAR1-selective compound” refers to a compound (e.g., compound described herein) having selectivity towards lysophosphatidic acid receptor 1 (LPAR1). In embodiments, the compound (e.g., compound described herein) is about 5-fold, 10-fold, 20-fold, 30-fold, 40-fold, 50-fold, or about 100-fold more selective for lysophosphatidic acid receptor 1 (LPAR1) over one or more of LPAR2, LPAR3, LPAR4, LPAR5, or LPAR6. In embodiments, the compound (e.g., compound described herein) is at least 5-fold, 10-fold, 20-fold, 30-fold, 40-fold, 50-fold, or at least 100-fold more selective for lysophosphatidic acid receptor 1 (LPAR1) over one or more of LPAR2, LPAR3, LPAR4, LPAR5, or LPAR6.

### II. Compounds

In an aspect is provided a compound, or a pharmaceutically acceptable salt or solvate thereof, having the formula:

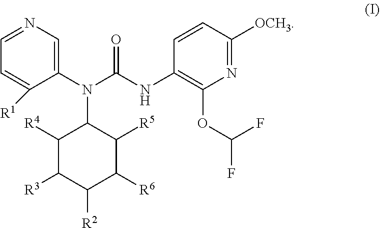

R¹ is isopropyl or isobutyl.
R² is —C(O)OH, —CH₂C(O)OH, —CH₂CH₂C(O)OH, —C(O)NHS(O)₂R^{2A}, —P(O)(OH)₂, —P(O)(OH)R^{2A}, —SO₃H, —SO₂H, —NHC(O)NHS(O)₂R^{2A},

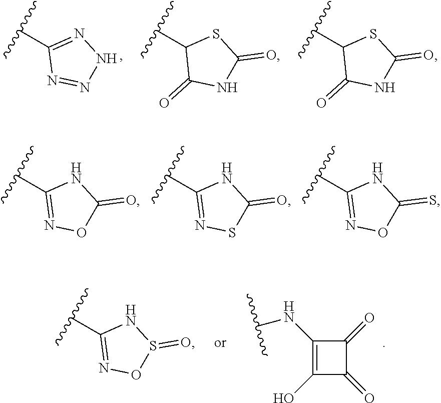

R^{2A} is unsubstituted C₁-C₄ alkyl.
R³, R⁴, R⁵, and R⁶ are independently hydrogen or unsubstituted C₁-C₄ alkyl.

In embodiments, the compound has the formula:

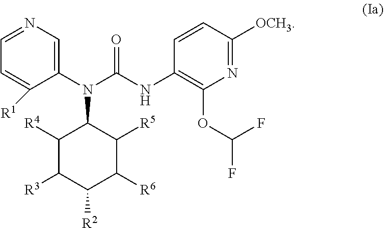

R¹, R², R³, R⁴, R⁵, and R⁶ are as described herein, including in embodiments.

In embodiments, the compound has the formula:

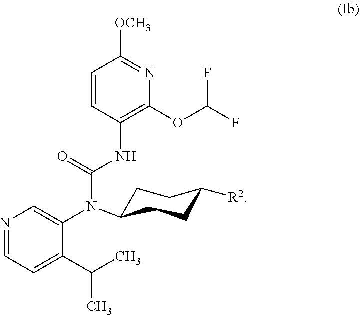

R² is as described herein, including in embodiments.

In an aspect is provided a compound, or a pharmaceutically acceptable salt or solvate thereof, having the formula:

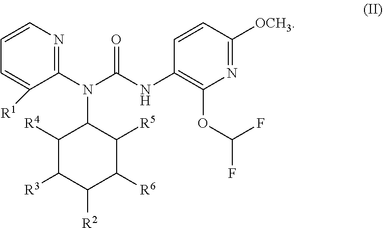

R¹, R², R³, R⁴, R⁵, and R⁶ are as described herein, including in embodiments.

In embodiments, the compound has the formula:

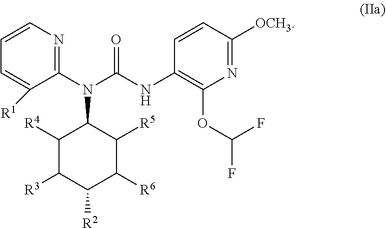

R¹, R², R³, R⁴, R⁵, and R⁶ are as described herein, including in embodiments.

In embodiments, the compound has the formula:

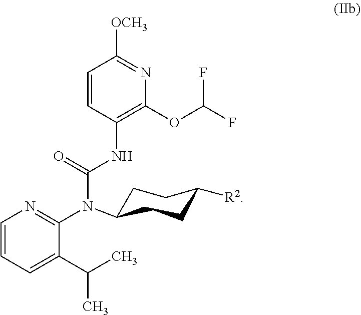

R² is as described herein, including in embodiments.

In embodiments, R¹ is isopropyl (i.e., unsubstituted isopropyl). In embodiments, R¹ is isobutyl (i.e., unsubstituted isobutyl).

In embodiments, R² is —C(O)OH. In embodiments, R² is —CH₂C(O)OH. In embodiments, R² is —CH₂CH₂C(O)OH. In embodiments, R² is —C(O)NHS(O)₂R^{2A}, wherein R^{2A} is as described herein, including in embodiments. In embodiments, R² is —C(O)NHS(O)₂CH₃. In embodiments, R² is —P(O)(OH)₂. In embodiments, R² is —P(O)(OH)R^{2A}, wherein R^{2A} is as described herein, including in embodiments. In embodiments, R² is —P(O)(OH)CH₃. In embodiments, R² is —SO₃H. In embodiments, R² is —SO₂H. In embodiments, R² is —NHC(O)NHS(O)₂R^{2A}, wherein R^{2A} is as described herein, including in embodiments. In embodiments, R² is —NHC(O)NHS(O)₂CH₃. In embodiments, R² is

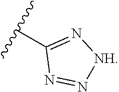 [SMILES: *c1nn[nH]n1]

In embodiments, R² is

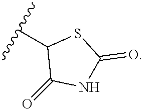 [SMILES: *C1SC(=O)NC1=O]

In embodiments, R² is

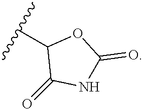 [SMILES: *C1OC(=O)NC1=O]

In embodiments, R² is

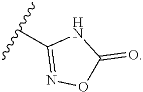 [SMILES: *c1noc(=O)[nH]1]

In embodiments, R² is

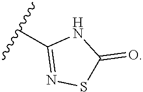 [SMILES: *c1nsc(=O)[nH]1]

In embodiments, R² is

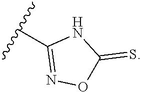 [SMILES: *c1noc(=S)[nH]1]

In embodiments, R² is

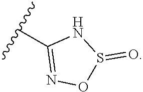 [SMILES: *C1=NOS(=O)N1]

In embodiments, R² is

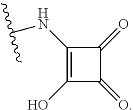 [SMILES: *Nc1c(O)c(=O)c1=O]

In embodiments, R^{2A} is unsubstituted methyl. In embodiments, R^{2A} is unsubstituted ethyl. In embodiments, R^{2A} is unsubstituted propyl. In embodiments, R^{2A} is unsubstituted n-propyl. In embodiments, R^{2A} is unsubstituted isopropyl. In embodiments, R^{2A} is unsubstituted butyl. In embodiments, R^{2A} is unsubstituted n-butyl. In embodiments, R^{2A} is unsubstituted isobutyl. In embodiments, R^{2A} is unsubstituted tert-butyl.

In embodiments, R³ is hydrogen or unsubstituted C₁-C₄ alkyl. In embodiments, R³ is hydrogen. In embodiments, R³ is unsubstituted methyl. In embodiments, R³ is unsubstituted ethyl. In embodiments, R³ is unsubstituted propyl. In embodiments, R³ is unsubstituted n-propyl. In embodiments, R³ is unsubstituted isopropyl. In embodiments, R³ is unsubstituted butyl. In embodiments, R³ is unsubstituted n-butyl. In embodiments, R³ is unsubstituted isobutyl. In embodiments, R³ is unsubstituted tert-butyl.

In embodiments, R⁴ is hydrogen or unsubstituted C₁-C₄ alkyl. In embodiments, R⁴ is hydrogen. In embodiments, R⁴ is unsubstituted methyl. In embodiments, R⁴ is unsubstituted ethyl. In embodiments, R⁴ is unsubstituted propyl. In embodiments, R⁴ is unsubstituted n-propyl. In embodiments, R⁴ is unsubstituted isopropyl. In embodiments, R⁴ is unsubstituted butyl. In embodiments, R⁴ is unsubstituted n-butyl. In embodiments, R⁴ is unsubstituted isobutyl. In embodiments, R⁴ is unsubstituted tert-butyl.

In embodiments, R⁵ is hydrogen or unsubstituted C₁-C₄ alkyl. In embodiments, R⁵ is hydrogen. In embodiments, R⁵ is unsubstituted methyl. In embodiments, R⁵ is unsubstituted ethyl. In embodiments, R⁵ is unsubstituted propyl. In embodiments, R⁵ is unsubstituted n-propyl. In embodiments, R⁵ is unsubstituted isopropyl. In embodiments, R⁵ is unsubstituted butyl. In embodiments, R⁵ is unsubstituted n-butyl. In embodiments, R⁵ is unsubstituted isobutyl. In embodiments, R⁵ is unsubstituted tert-butyl.

In embodiments, R⁶ is hydrogen or unsubstituted C₁-C₄ alkyl. In embodiments, R⁶ is hydrogen. In embodiments, R⁶ is unsubstituted methyl. In embodiments, R⁶ is unsubstituted ethyl. In embodiments, R⁶ is unsubstituted propyl. In embodiments, R⁶ is unsubstituted n-propyl. In embodiments, R⁶ is unsubstituted isopropyl. In embodiments, R⁶ is unsubstituted butyl. In embodiments, R⁶ is unsubstituted n-butyl. In embodiments, R⁶ is unsubstituted isobutyl. In embodiments, R⁶ is unsubstituted tert-butyl.

In embodiments, R³, R⁴, R⁵, and R⁶ are independently hydrogen or unsubstituted methyl.

In embodiments, the compound has the formula:

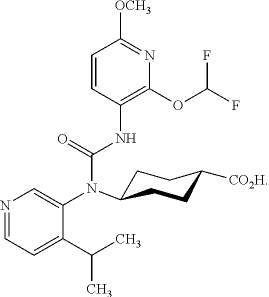

In embodiments, the compound has the formula:

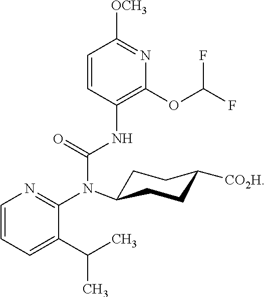

In embodiments, the compound is useful as a comparator compound. In embodiments, the comparator compound can be used to assess the activity of a test compound as set forth in an assay described herein (e.g., in the examples section, figures, or tables).

In embodiments, the compound is a compound as described herein, including in embodiments. In embodiments the compound is a compound described herein (e.g., in the examples section, figures, tables, or claims).

In embodiments, the compound does not significantly cross the blood-brain barrier. In embodiments, the compound does not detectably cross the blood-brain barrier. In embodiments, the compound does not cross the blood-brain barrier. In embodiments, the compound has a K_{p,uu} of less than about 0.2, wherein K_{p,uu} is the unbound brain-to-plasma partition coefficient, and describes the unbound concentration of the compound in the brain relative to the plasma. In embodiments, the compound has a K_{p,uu} of less than about 0.18. In embodiments, the compound has a K_{p,uu} of less than about 0.16. In embodiments, the compound has a K_{p,uu} of less than about 0.14. In embodiments, the compound has a K_{p,uu} of less than about 0.12. In embodiments, the compound has a K_{p,uu} of less than about 0.1. In embodiments, the compound has a K_{p,uu} of less than about 0.08. In embodiments, the compound has a K_{p,uu} of less than about 0.06. In embodiments, the compound has a K_{p,uu} of less than about 0.04. In embodiments, the compound has a K_{p,uu} of less than about 0.02. In embodiments, the compound has a K_{p,uu} of less than 0.2. In embodiments, the compound has a K_{p,uu} of less than 0.18. In embodiments, the compound has a K_{p,uu} of less than 0.16. In embodiments, the compound has a K_{p,uu} of less than 0.14. In embodiments, the compound has a K_{p,uu} of less than 0.12. In embodiments, the compound has a K_{p,uu} of less than 0.1. In embodiments, the compound has a K_{p,uu} of less than 0.08. In embodiments, the compound has a K_{p,uu} of less than 0.06. In embodiments, the compound has a K_{p,uu} of less than 0.04. In embodiments, the compound has a K_{p,uu} of less than 0.02.

### III. Pharmaceutical Compositions

In an aspect is provided a pharmaceutical composition including a compound described herein, or a pharmaceutically acceptable salt or solvate thereof, and a pharmaceutically acceptable excipient.

In embodiments, the compound is a compound of formula (I), (Ia), (Ib), (II), (IIa), or (IIb), including embodiments thereof.

In embodiments, the pharmaceutical composition includes an effective amount of the compound. In embodiments, the pharmaceutical composition includes a therapeutically effective amount of the compound.

### IV. Methods of Use

In an aspect is provided a method of treating a peripherally restricted LPAR1-associated disease in a subject in need thereof, the method including administering to the subject a therapeutically effective amount of a compound described herein, or a pharmaceutically acceptable salt of solvate thereof.

In embodiments, the compound is a compound of formula (I), (Ia), (Ib), (II), (IIa), or (IIb), including embodiments thereof.

In embodiments, the peripherally restricted LPAR1-associated disease is a fibrotic disease. In embodiments, the fibrotic disease is pulmonary fibrosis. In embodiments, the fibrotic disease is idiopathic pulmonary fibrosis. In embodiments, the fibrotic disease is renal fibrosis. In embodiments, the fibrotic disease is skin fibrosis. In embodiments, the fibrotic disease is ocular fibrosis. In embodiments, the fibrotic disease is nonalcoholic steatohepatitis. In embodiments, the fibrotic disease is scleroderma. In embodiments, the fibrotic disease is hypertrophic scarring or keloids (e.g., burn induced or surgical, sarcoidosis, scleroderma, spinal cord injury/fibrosis, myelofibrosis, vascular restenosis, atherosclerosis, arteriosclerosis, Wegener's granulomatosis, mixed connective tissue disease, or Peyronie's disease). In embodiments, the fibrotic disease is iatrogenic pulmonary fibrosis. In embodiments, the fibrotic disease is radiation-induced fibrosis. In embodiments, the fibrotic disease is silicosis-induced pulmonary fibrosis. In embodiments, the fibrotic disease is asbestos-induced pulmonary fibrosis. In embodiments, the fibrotic disease is pleural fibrosis. In embodiments, the fibrotic disease is pulmonary fibrosis associated with SARS-CoV-2 infection and/or COVID-19. In embodiments, the fibrotic disease is pulmonary fibrosis secondary to systemic inflammatory disease. In embodiments, the fibrotic disease is pulmonary fibrosis secondary to sarcoidosis. In embodiments, the fibrotic disease is gut fibrosis. In embodiments, the fibrotic disease is head and neck fibrosis. In embodiments, the fibrotic disease is cirrhosis. In embodiments, the fibrotic disease is alcohol-induced liver fibrosis. In embodiments, the fibrotic disease is endometriosis. In embodiments, the fibrotic disease is spinal cord fibrosis. In embodiments, the fibrotic disease is myelofibrosis. In embodiments, the fibrotic disease is cardiac fibrosis. In embodiments, the fibrotic disease is perivascular fibrosis. In embodiments, the fibrotic disease is Peyronie's disease. In embodiments, the fibrotic disease is abdominal or bowel adhesions. In embodiments, the fibrotic disease is bladder fibrosis. In embodiments, the fibrotic disease is fibrosis of the nasal passages. In embodiments, the fibrotic disease is fibrosis mediated by fibroblasts. In embodiments, the fibrotic disease is renal fibrosis associated with chronic kidney disease (CKD). In embodiments, the fibrotic disease is intestinal fibrosis. In embodiments, the fibrotic disease is fibrosis of the gastrointestinal tract.

In embodiments, the peripherally restricted LPAR1-associated disease is a cancer. In embodiments, the cancer is ovarian cancer. In embodiments, the cancer is breast cancer. In embodiments, the cancer is liver cancer. In embodiments, the cancer is gastric cancer. In embodiments, the cancer is pancreatic cancer. In embodiments, the cancer is lung cancer. In embodiments, the cancer is osteosarcoma.

In embodiments, the peripherally restricted LPAR1-associated disease is inflammatory bowel disease. In embodiments, the inflammatory bowel disease is a chronic inflammatory bowel disease. In embodiments, the inflammatory bowel disease is ulcerative colitis. In embodiments, the inflammatory bowel disease is Crohn's disease. In embodiments, the inflammatory bowel disease is intestinal fibrosis. In embodiments, the inflammatory bowel disease is fibrosis of the gastrointestinal tract.

In an aspect is provided a method of modulating LPAR1 activity in a subject, the method including administering to the subject a compound described herein, or a pharmaceutically acceptable salt or solvate thereof.

### V. Embodiments

Embodiment P1. A compound, or a pharmaceutically acceptable salt or solvate thereof, having the formula:

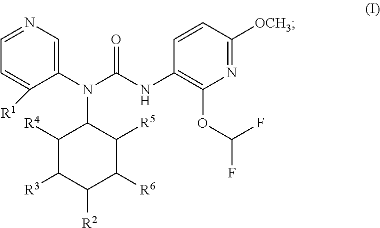

wherein
R¹ is isopropyl or isobutyl;
R² is —C(O)OH, —CH₂C(O)OH, —CH₂CH₂C(O)OH, —C(O)NHS(O)₂R^{2A}, —P(O)(OH)₂, —P(O)(OH)R^{2A}, —SO₃H, —SO₂H, —NHC(O)NHS(O)₂R^{2A},

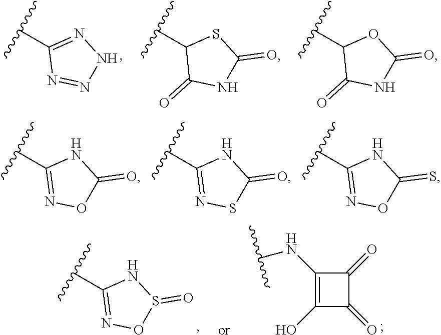

R^{2A} is unsubstituted C₁-C₄ alkyl; and
R³, R⁴, R⁵, and R⁶ are independently hydrogen or unsubstituted C₁-C₄ alkyl.
Embodiment P2. The compound of embodiment P1, wherein R³, R⁴, R⁵, and R⁶ are independently hydrogen or unsubstituted methyl.
Embodiment P3. The compound of embodiment P1, having the formula:

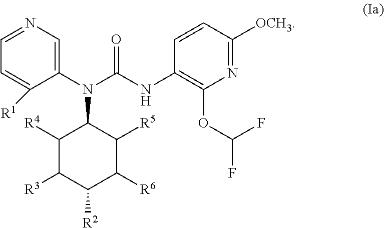

Embodiment P4. The compound of embodiment P1, having the formula:

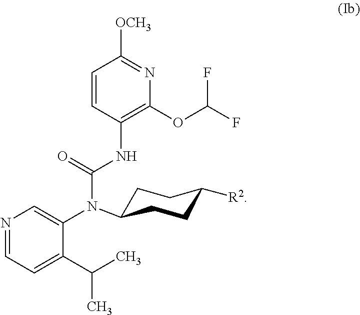

Embodiment P5. The compound of one of embodiments P1 to P4, wherein R² is —C(O)OH, —CH₂C(O)OH, —CH₂CH₂C(O)OH, —C(O)NHS(O)₂CH₃, —P(O)(OH)₂, —P(O)(OH)CH₃, —SO₃H, —SO₂H, —NHC(O)NHS(O)₂CH₃,

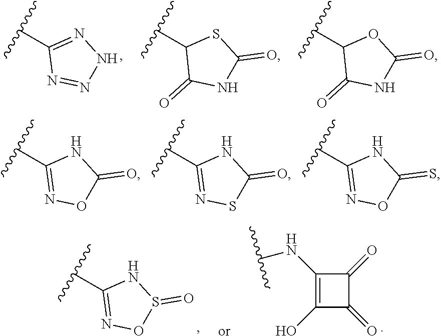

Embodiment P6. The compound of one of embodiments P1 to P4, wherein R² is —C(O)OH.
Embodiment P7. The compound of embodiment P1, having the formula:

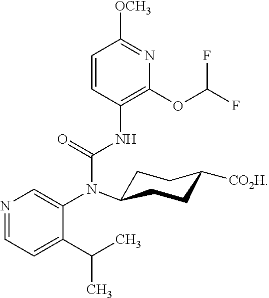

Embodiment P8. A compound, or a pharmaceutically acceptable salt or solvate thereof, having the formula:

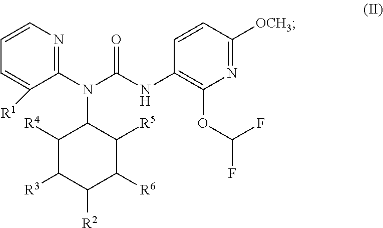

wherein
R¹ is isopropyl or isobutyl;
R² is —C(O)OH, —CH₂C(O)OH, —CH₂CH₂C(O)OH, —C(O)NHS(O)₂R^{2A}, —P(O)(OH)₂, —P(O)(OH)R^{2A}, —SO₃H, —SO₂H, —NHC(O)NHS(O)₂R^{2A},

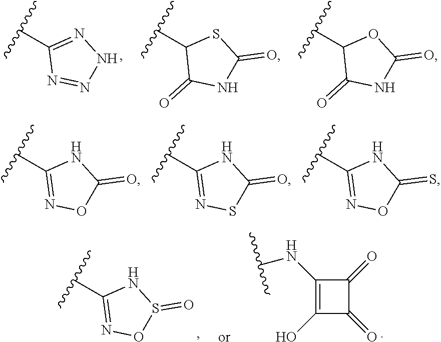

R^{2A} is unsubstituted C₁-C₄ alkyl; and
R³, R⁴, R⁵, and R⁶ are independently hydrogen or unsubstituted C₁-C₄ alkyl.

Embodiment P9. The compound of embodiment P8, wherein R³, R⁴, R⁵, and R⁶ are independently hydrogen or unsubstituted methyl.

Embodiment P10. The compound of embodiment P8, having the formula:

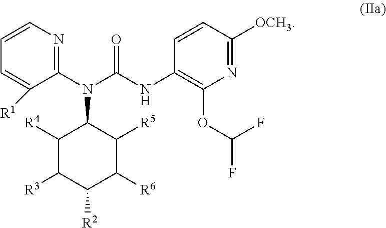

Embodiment P11. The compound of embodiment P8, having the formula:

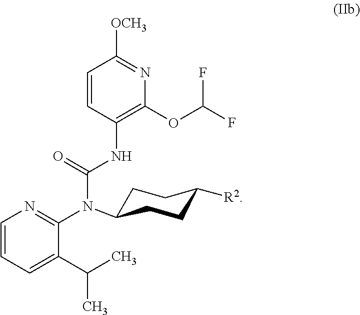

Embodiment P12. The compound of one of embodiments P8 to P11, wherein R² is —C(O)OH, —CH₂C(O)OH, —CH₂CH₂C(O)OH, —C(O)NHS(O)₂CH₃, —P(O)(OH)₂, —P(O)(OH)CH₃, —SO₃H, —SO₂H, —NHC(O)NHS(O)₂CH₃,

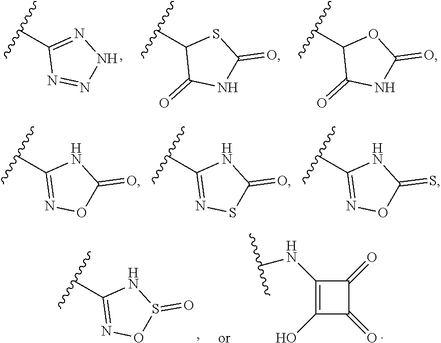

Embodiment P13. The compound of one of embodiments P8 to P11, wherein R² is —C(O)OH.
Embodiment P14. The compound of one of embodiments P1 to P13, having the formula:

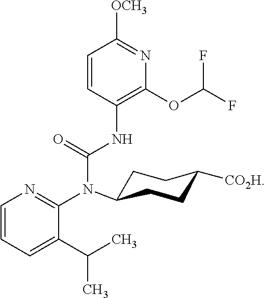

Embodiment P15. A pharmaceutical composition comprising the compound of one of embodiments P1 to P14, or a pharmaceutically acceptable salt or solvate thereof, and a pharmaceutically acceptable excipient.
Embodiment P16. A method of treating a peripherally restricted LPAR1-associated disease in a subject in need thereof, said method comprising administering to the subject a therapeutically effective amount of the compound of one of embodiments P1 to P14, or a pharmaceutically acceptable salt or solvate thereof.
Embodiment P17. The method of embodiment P16, wherein the peripherally restricted LPAR1-associated disease is a fibrotic disease.
Embodiment P18. The method of embodiment P17, wherein the fibrotic disease is idiopathic pulmonary fibrosis, renal fibrosis, nonalcoholic steatohepatitis, or scleroderma.
Embodiment P19. The method of embodiment P16, wherein the peripherally restricted LPAR1-associated disease is a cancer.
Embodiment P20. The method of embodiment P19, wherein the cancer is ovarian cancer, breast cancer, liver cancer, gastric cancer, pancreatic cancer, lung cancer, or osteosarcoma.
Embodiment P21. A method of modulating LPAR1 activity in a subject, said method comprising administering to the subject a compound of one of embodiments P1 to P14, or a pharmaceutically acceptable salt or solvate thereof.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### 1. Preparation of the Compounds

The compounds used in the reactions described herein are made according to known organic synthesis techniques, starting from commercially available chemicals and/or from compounds described in the chemical literature. “Commercially available chemicals” are obtained from standard commercial sources including Acros Organics (Geel, Belgium), Aldrich Chemical (Milwaukee, WI, including Sigma Chemical and Fluka), Apin Chemicals Ltd. (Milton Park, UK), Ark Pharm, Inc. (Libertyville, IL), Avocado Research (Lancashire, U.K.), BDH Inc. (Toronto, Canada), Bionet (Cornwall, U.K.), Chemservice Inc. (West Chester, PA), Combi-blocks (San Diego, CA), Crescent Chemical Co. (Hauppauge, NY), eMolecules (San Diego, CA), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Cornwall, U.K.), Lancaster Synthesis (Windham, NH), Matrix Scientific, (Columbia, SC), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Ryan Scientific, Inc. (Mount Pleasant, SC), Spectrum Chemicals (Gardena, CA), Sundia Meditech, (Shanghai, China), TCI America (Portland, OR), Trans World Chemicals, Inc. (Rockville, MD), and WuXi (Shanghai, China).

Suitable reference books and treatises that detail the synthesis of reactants useful in the preparation of compounds described herein, or provide references to articles that describe the preparation, include for example, “Synthetic Organic Chemistry,” New York: John Wiley & Sons, Inc., 1982; Sandler S. R. et al., “Organic Functional Group Preparations,” 2^{nd} ed., New York: Academic Press, 1983; House, H. O., “Modern Synthetic Reactions,” 2^{nd} ed., Menlo Park: W. A. Benjamin, Inc., 1972; Gilchrist, T. L., “Heterocyclic Chemistry,” 2^{nd} ed., New York: Wiley, 1992; March, J., “Advanced Organic Chemistry: Reactions, Mechanisms and Structure,” 4^{th} ed., New York: Wiley, 1992. Additional suitable reference books and treatises that detail the synthesis of reactants useful in the preparation of compounds described herein, or provide references to articles that describe the preparation, include for example, Fuhrhop, J., Penzlin, G., “Organic Synthesis: Concepts, Methods, Starting Materials,” 2^{nd} ed., New York: Wiley, 1994; Hoffman, R. V., “Organic Chemistry, An Intermediate Text,” Oxford: Oxford University Press, 1996; Larock, R. C., “Comprehensive Organic Transformations: A Guide to Functional Group Preparations,” 2^{nd} ed., New York: Wiley, 1999; Otera, J., “Modern Carbonyl Chemistry,” New York: Wiley, 2000; Solomons, T. W. G., “Organic Chemistry,” 7^{th} ed., New York: Wiley, 2000; Stowell, J. C., “Intermediate Organic Chemistry,” 2^{nd} ed., New York: Wiley, 1993; “Industrial Organic Chemicals: Starting Materials and Intermediates: An Ullmann's Encyclopedia,” New York: Wiley, in 8 volumes; “Organic Reactions,” New York: Wiley, in over 55 volumes; and “Chemistry of Functional Groups,” New York: Wiley, in 73 volumes.

Specific and analogous reactants are also identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is Stahl, P. H., Wermuth, C. G., “Handbook of Pharmaceutical Salts,” Zurich: Verlag Helvetica Chimica Acta, 2002.

### List of Abbreviations

As used above, and throughout the description of the application, the following abbreviations, unless otherwise indicated, shall be understood to have the following meanings:
ACN or MeCN acetonitrile
aq aqueous
BSA bovine serum albumin
CDI 1,1′-carbonyldiimidazole
CHO Chinese hamster ovary
cPhos 2-dicyclohexylphosphino-2′,6′-bis(N,N-dimethylamino)biphenyl
d doublet
dd doublet of doublets
dba dibenzylideneacetone
DBU 1,8-diazabicyclo[5.4.0]undec-7-ene
DCE dichloroethane (ClCH₂CH₂Cl)
DCM dichloromethane (CH₂Cl₂)
DIPEA or DIEA N,N-diisopropylethylamine
DMAP 4-(N,N-dimethylamino)pyridine
DMEM Dulbecco's modified eagle medium
DMSO dimethylsulfoxide
EDC N-(3-dimethylaminopropyl)-N′-ethylcarbodiimide
equiv equivalent(s)
Et ethyl
EtOAc ethyl acetate
FBS fetal bovine serum
h hour(s)
Hal halogen
HATU 1-[bis(dimethylamino)methylene]-1H-1,2,3-triazolo[4,5-b]pyridinium 3-oxide hexafluorophosphate
HEPES 4-(2-hydroxyethyl)-1-piperazineethanesulfonic acid
Hex hexanes
LCMS or LC-MS liquid chromatography-mass spectrometry
m multiplet
M molar or metal
Me methyl
MeOH methanol
min minute(s)
MS mass spectroscopy
NMI N-methylimidazole
NMR nuclear magnetic resonance
Pd/C palladium on carbon
RT room temperature
sept septet
t triplet
T3P propylphosphonic anhydride
TEA triethylamine
THF tetrahydrofuran
v/v volume per volume
w/w weight per weight

### General Synthetic Schemes

Compounds of Formula (I) or Formula (II) of the present disclosure may be prepared, respectively, from the union of amine (1) with a secondary heteroaryl amine (2a) or (2b), in the presence of an appropriate “C═O” source such as CDI, phosgene, triphosgene, or the like, and an appropriate base such as pyridine, TEA, DIEA, sodium hydride, or the like (Scheme 1). Depending on the reactivity of the two amine coupling partners, it may be advantageous to first react amine (1) with the aforementioned “C═O” source and base, to deliver the requisite carbamoyl chloride, carbamoyl imidazole, or isocyanate intermediate, prior to the addition of the other amine coupling partner, or vice versa. In some cases, the formation of the final urea bond can be promoted with the addition of activators such as DMAP, freshly activated molecular sieves, heat, or the like.

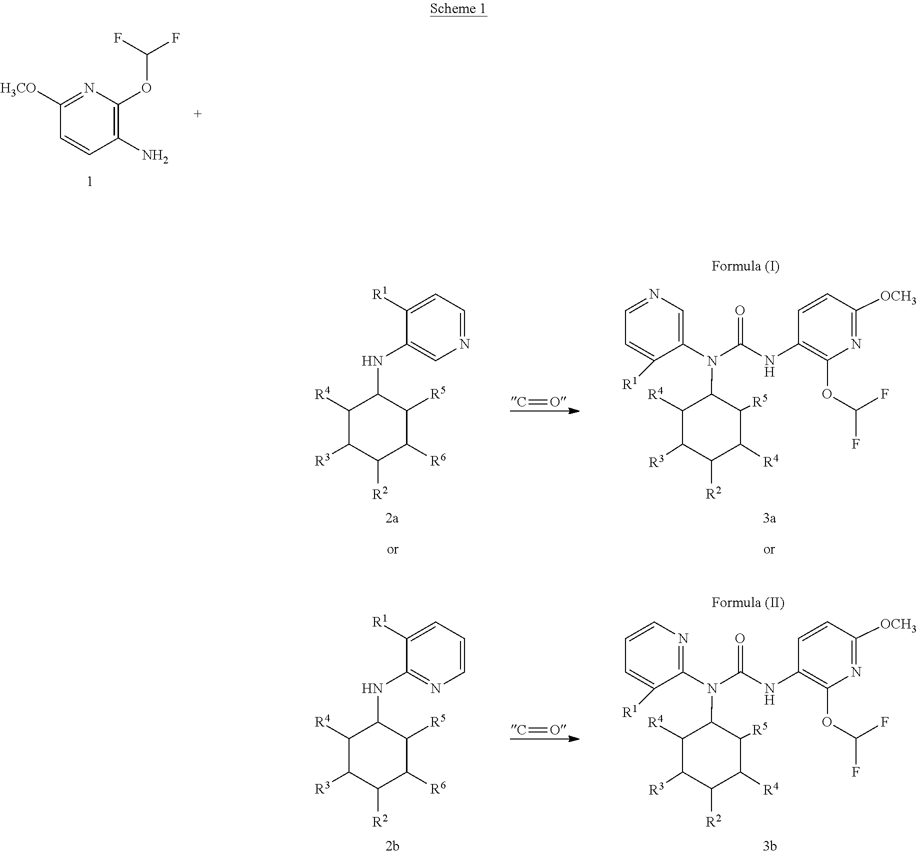

The product urea (3a) or (3b), which itself may be a compound of Formula (I) or Formula (II), can be further functionalized using synthetic methodologies known to those skilled in the art to deliver another compound of Formula (I) or Formula (II). Examples of such transformations include, but are not limited to:
(a) hydrolysis of an ester present in (3a) or (3b), with a suitable reagent such as lithium hydroxide, sodium hydroxide, or potassium hydroxide, or the like.
(b) hydrolysis of a nitrile present in (3a) or (3b), with a suitable reagent such as wet sulfuric acid, wet hydrochloric acid, lithium hydroxide, sodium hydroxide, or the like.
(c) cycloaddition of a nitrile present in (3a) or (3b), with a suitable azide such as trimethylsilyl azide, sodium azide, tetrabutylammonium azide, or the like, in the presence of a suitable promoter such as a dibutyltin(IV) oxide, protic acid, heat, or the like.
(d) capping of an unmasked amine present in (3a) or (3b), revealed following the removal of the masking group using conditions known to those skilled in the art, with a suitable N-sulfonyl isocyanate, N-sulfonyl carbamate, or the like, in the presence of a suitable base such as TEA, pyridine, sodium hydride, cesium carbonate, or the like.
(e) coupling of a carboxylic acid present in (3a) or (3b), with an appropriately functionalized sulfonamide, in the presence of a suitable coupling reagent such as HATU, CDI, T3P, EDC, or the like, and a suitable base such as TEA, DIEA, NMI, or the like.
(f) initial activation of a carboxylic acid present in (3a) or (3b) as an acid chloride or an anhydride, using conditions known to those skilled in the art, followed by quenching with an appropriately functionalized sulfonamide in the presence of a suitable base such as TEA, DIEA, NMI, or the like.
(g) hydrolysis of a dialkyl phosphonate or an alkyl phosphinate present in (3a) or (3b), with a suitable reagent such aqueous HCl, aqueous sodium hydroxide, or the like, or using the two-step McKenna procedure where the aforementioned dialkyl phosphonate or alkyl phosphinate is first treated with bromotrimethyl silane, and then with wet methanol, or the like.
(h) initial oxidation of a thioacetate or a thioether present in (3a) or (3b) with tert-butyl hypochlorite, N-chlorosuccinimide, or the like, followed by quenching with water.
(i) initial oxidation of a thioacetate or a thioether present in (3a) or (3b) with tert-butyl hypochlorite, N-chlorosuccinimide, or the like, followed by reduction with aqueous sodium sulfite, or the like.
(j) initial activation of an alcohol present in (3a) or (3b) as an alkyl halide or an alkyl triflate, using conditions known to those skilled in the art, followed by quenching with a lithiated thiazolidin-2,4-dione, oxazolidine-2,4-dione, or the like.
(k) initial conversion of a nitrile present in (3a) or (3b) into the corresponding N-hydroxyl imidamide, using conditions known to those skilled in the art, followed by quenching with the appropriate acylating agent such as isobutyl chloroformate, 1,1′-thiocarbonyldiimidazole, thionyl chloride, or the like, in the presence of a suitable promoter such as heat, boron trifluoride etherate, DBU, or the like.
(l) heating of an unmasked amine present in (3a) or (3b), revealed following the removal of the masking group using conditions known to those skilled in the art, with squaric acid.
(m) separation of a mixture of stereoisomers into its stereochemically-enriched constituents utilizing an appropriate chiral column such as ChiralPAK IF, CHIRAL ART Amylose SA, CHIRAL ART Cellulose SB, or the like.

For certain embodiments, a person skilled in the art can access the secondary heteroaryl amine (2a) or (2b) used for the coupling depicted in Scheme 1 by the union of an appropriately functionalized amine (4) with an appropriately functionalized heteroaryl halide (5a) or (5b), in the presence of a promoter such as a palladium catalyst, a copper catalyst, heat, or the like, and a base such as potassium phosphate, TEA, potassium bis(trimethylsilyl)amide, or the like (Scheme 2).

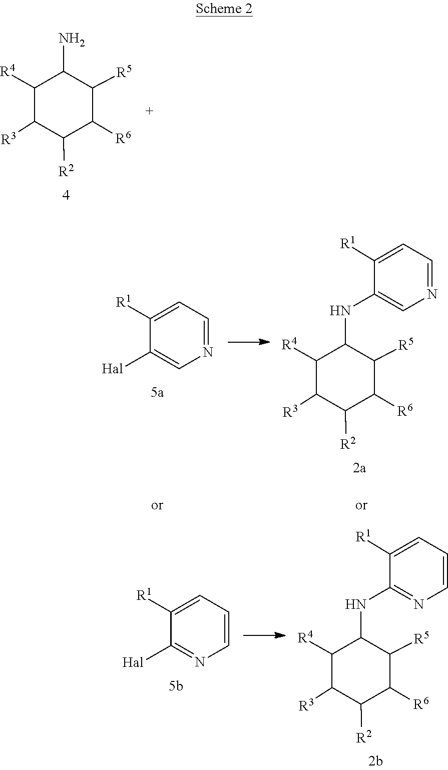

For certain embodiments, the secondary heteroaryl amine (2a) or (2b) used for the coupling depicted in Scheme 1 can instead be accessed from the reductive amination of an appropriately functionalized ketone (6) with an appropriately functionalized heteroaryl amine (7a) or (7b), in the presence of a suitable a reducing agent such as sodium cyanoborohydride, sodium triacetoxyborohydride, or the like, and a suitable additive such as acetic acid, titanium(IV) isopropoxide, or the like (Scheme 3).

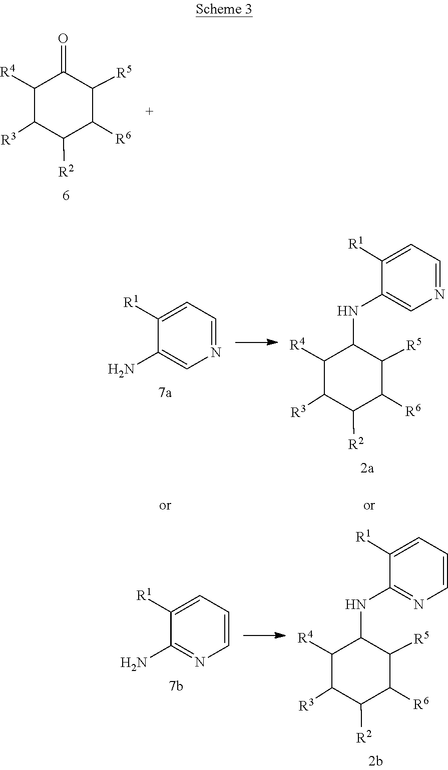

For certain embodiments, due to, for example, greater accessibility of the requisite starting materials or greater facility with which the requisite bond formation proceeds, it may be advantageous to delay the introduction of the R¹ group. In these instances, the secondary halo-heteroaryl amine (9a) or (9b), itself synthesized from the corresponding halo-heteroarene (8a) or (8b), using the strategies disclosed previously, can be converted to the secondary heteroaryl amine (2a) or (2b), by its metal-catalyzed cross coupling with an appropriately functionalized organometallic reagent (10), using conditions known to those skilled in the art (Scheme 4). In certain cases, the conversion of (9a) to (2a), or of (9b) to (2b), can require a two-step sequence involving an initial metal-catalyzed cross coupling with (10), followed by a hydrogenation of the intermediate alkene or alkyne.

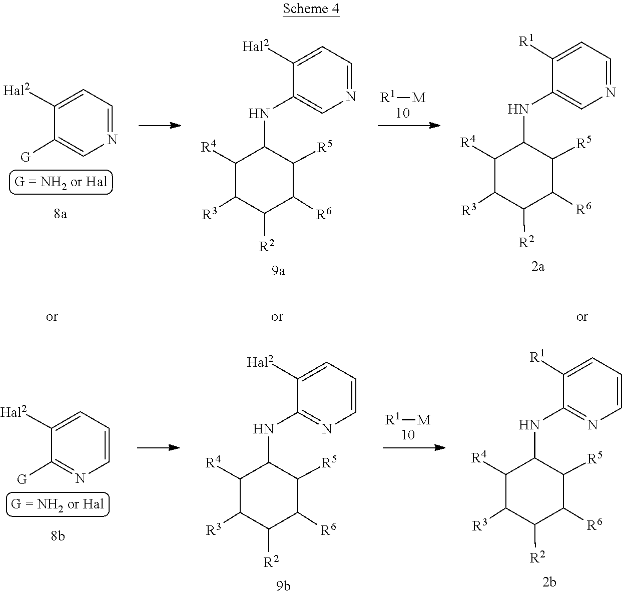

The general synthetic schemes above have been described in an illustrative manner and is intended to be in the nature of description rather than of limitation. It will also be appreciated that many of the reagents provided in the following examples may be substituted with other suitable reagents (see, e.g., is Fieser, L., et al., “Encyclopedia of Reagents for Organic Synthesis,” 2^{nd} ed., New York: Wiley, 2009). In addition, it will be appreciated that conditions such as choice of solvent, temperature of reaction, volumes and reaction time may vary while still producing the desired compounds. Such changes and modifications, including without limitation, those relating to the chemical structures, substituents, derivatives, intermediates and/or syntheses provided herein, may be made without departing from the spirit and scope thereof.

### EXAMPLES

Unless otherwise noted, reagents and solvents were used as received from commercial suppliers. Anhydrous solvents and oven-dried glassware were used for synthetic transformations sensitive to moisture and/or oxygen. Yields were not optimized. Reaction times are approximate and were not optimized. Column chromatography was performed on silica gel unless otherwise noted.

### Intermediate amine 1: Preparation of trans-ethyl 4-((4-isopropylpyridin-3-yl)amino)cyclohexane-1-carboxylate

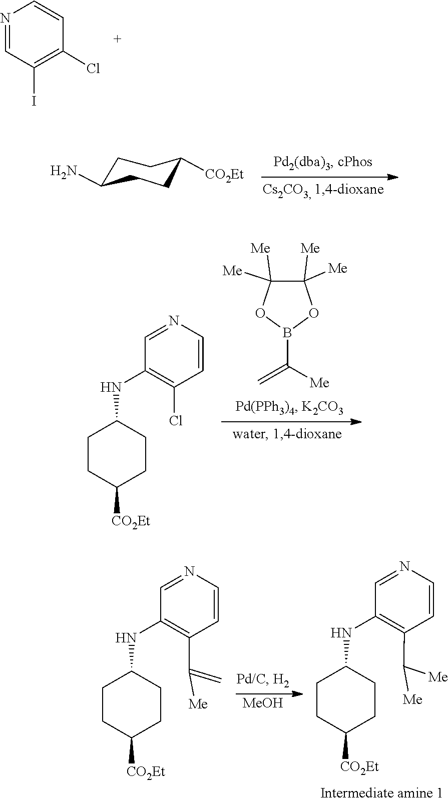

Step 1: In a thick-walled glass reaction vessel equipped with a magnetic stirrer and a Teflon screwcap was combined trans-ethyl 4-aminocyclohexanecarboxylate (1 equiv), 4-chloro-3-iodopyridine (1.5 equiv), tris(dibenzylidineacetone)dipalladium(0) (0.1 equiv), 2-dicyclohexylphosphino-2′,6′-bis(N,N-dimethylamino)biphenyl (0.2 equiv), and cesium carbonate (3 equiv) in 1,4-dioxane (0.73 M). The resulting purple suspension was then deoxygenated via subsurface purging with nitrogen for 10 min before the reaction vessel was tightly sealed and heated at 100° C. for 60 h. The resulting dark brown suspension was cooled to RT, diluted with EtOAc, and washed sequentially with water and brine. The organic extract thus obtained was then dried over Na₂SO₄, filtered, and the filtrate concentrated in vacuo. Purification of the crude product thus obtained by way of column chromatography (SiO₂, gradient elution: 9:1 (v/v) Hex:EtOAc→EtOAc) afforded trans-ethyl 4-((4-chloropyridin-3-yl)amino)cyclohexane-1-carboxylate as a yellow oil (74% yield).

Step 2: In a thick-walled glass reaction vessel equipped with a magnetic stirrer and a Teflon screwcap was combined trans-ethyl 4-((4-chloropyridin-3-yl)amino)cyclohexane-1-carboxylate (1 equiv) from the previous step, 4,4,5,5-tetramethyl-2-(prop-1-en-2-yl)-1,3,2-dioxaborolane (2 equiv), tetrakis(triphenylphosphine)palladium(0) (0.2 equiv), and potassium carbonate (3 equiv) in a 5:1 (v/v) solution of 1,4-dioxane and water (0.15 M). The resulting mixture was deoxygenated via subsurface purging with nitrogen for 10 min before the reaction vessel was tightly sealed and heated at 90° C. for 72 h. The now dark brown suspension was cooled to RT, quenched with water, and extracted with EtOAc. The combined organic extracts were washed further with water and brine, dried over Na₂SO₄, filtered, and the filtrate concentrated in vacuo. Purification of the crude product thus obtained by way of column chromatography (SiO₂, gradient elution: 9:1 (v/v) Hex:EtOAc→EtOAc) afforded trans-ethyl 4-((4-(prop-1-en-2-yl)pyridin-3-yl)amino)cyclohexane-1-carboxylate as a yellow oil (96% yield).

Step 3: In a dried, round-bottom flask equipped with a magnetic stirrer was dissolved trans-ethyl 4-((4-(prop-1-en-2-yl)pyridin-3-yl)amino)cyclohexane-1-carboxylate (1 equiv) from the previous step in methanol (0.11 M). The resulting solution was then deoxygenated via subsurface purging with nitrogen for 10 min before palladium (10% w/w over activated carbon, dry, 0.1 equiv) was added in one rapid portion. The resulting black suspension was then subsurface purged with hydrogen for 10 min before it was stirred under a static hydrogen atmosphere (maintained with a balloon) at RT for 24 h. The reaction was subsequently quenched with dichloromethane and filtered through a bed of dichloromethane-wetted celite. Concentration of the filtrate in vacuo afforded the title compound as a yellow oil (83% yield).

### Intermediate amine 2: Preparation of trans-ethyl 4-((3-isopropylpyridin-2-yl)amino)cyclohexane-1-carboxylate

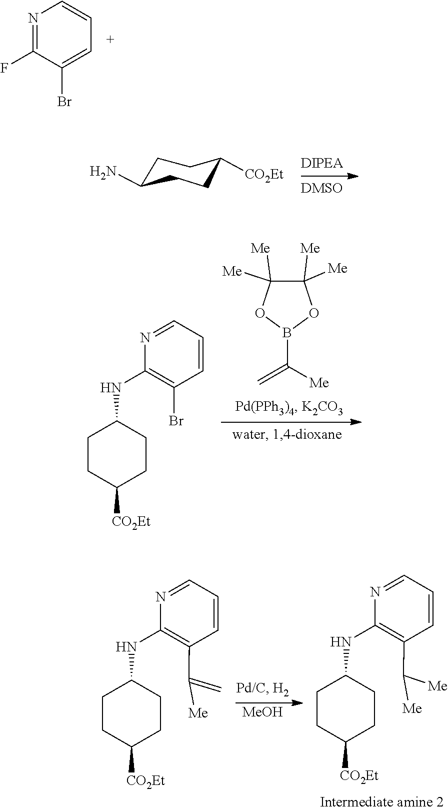

Step 1: In a dried, round-bottom flask equipped with a magnetic stirrer was combined trans-ethyl 4-aminocyclohexanecarboxylate (1 equiv), 3-bromo-2-fluoropyridine (1.5 equiv), and N,N-diisopropylethylamine (3 equiv) in dimethylsulfoxide (0.78 M). The resulting solution was then heated at 120° C. for 16 h. After cooling to RT, the reaction was quenched with water and extracted with EtOAc. The combined organic extracts were then washed further with water and brine, dried over Na₂SO₄, filtered, and the filtrate concentrated in vacuo. Purification of the crude product thus obtained by way of column chromatography (SiO₂, gradient elution: 9:1 (v/v) Hex:EtOAc→EtOAc) afforded trans-ethyl 4-((3-bromopyridin-2-yl)amino)cyclohexane-1-carboxylate as a white solid (83% yield).

Step 2: In a thick-walled glass reaction vessel equipped with a magnetic stirrer and a Teflon screwcap was combined trans-ethyl 4-((3-bromopyridin-2-yl)amino)cyclohexane-1-carboxylate (1 equiv) from the previous step, 4,4,5,5-tetramethyl-2-(prop-1-en-2-yl)-1,3,2-dioxaborolane (2 equiv), tetrakis(triphenylphosphine)palladium(0) (0.2 equiv), and potassium carbonate (3 equiv) in a 5:1 (v/v) solution of 1,4-dioxane and water (0.08 M). The resulting mixture was deoxygenated via subsurface purging with nitrogen for 10 min before the reaction vessel was tightly sealed and heated at 90° C. for 16 h. The now dark brown suspension was cooled to RT, quenched with water, and extracted with EtOAc. The combined organic extracts were washed further with water and brine, dried over Na₂SO₄, filtered, and the filtrate concentrated in vacuo. Purification of the crude product thus obtained by way of column chromatography (SiO₂, gradient elution: 9:1 (v/v) Hex:EtOAc→EtOAc) afforded trans-ethyl 4-((3-(prop-1-en-2-yl)pyridin-2-yl)amino)cyclohexane-1-carboxylate as a yellow oil (68% yield).

Step 3: In a dried, round-bottom flask equipped with a magnetic stirrer was dissolved trans-ethyl 4-((3-(prop-1-en-2-yl)pyridin-2-yl)amino)cyclohexane-1-carboxylate (1 equiv) from the previous step in methanol (0.19 M). The resulting solution was then deoxygenated via subsurface purging with nitrogen for 10 min before palladium (10% w/w over activated carbon, dry, 0.1 equiv) was added in one rapid portion. The resulting black suspension was then subsurface purged with hydrogen for 10 min before it was stirred under a static hydrogen atmosphere (maintained with a balloon) at RT for 1 h. The reaction was subsequently quenched with dichloromethane and filtered through a bed of dichloromethane-wetted celite. Concentration of the filtrate in vacuo afforded the title compound as a yellow oil (92% yield).

### Intermediate amine 3: Preparation of 2-(difluoromethoxy)-6-methoxypyridin-3-amine

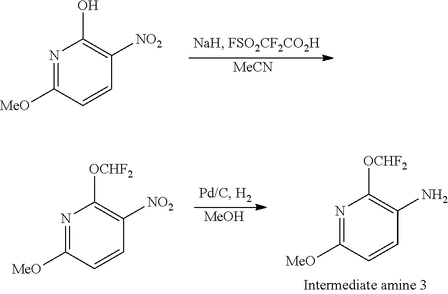 [SMILES: "MeCN" ; "MeOH" ; COc1ccc([N+](=O)[O-])c(O)n1 ; COc1ccc([N+](=O)[O-])c(OC(F)F)n1 ; COc1ccc(N)c(OC(F)F)n1]

Step 1: In a dried, round-bottom flask equipped with a magnetic stirrer was suspended 6-methoxy-3-nitropyridin-2-ol (1 equiv) in acetonitrile (0.10 M). To this was then added sodium hydride (60% w/w dispersion in paraffin oil, 2.8 equiv) in one rapid portion and the resulting mixture was stirred at RT for 10 min to afford a brownish, yellow suspension. Then, 2,2-difluoro-2-(fluorosulfonyl)acetic acid (1.8 equiv) was added neat and dropwise over a period of 5 min, during which time a mild exotherm was observed. After 16 h of stirring, another aliquot of 2,2-difluoro-2-(fluorosulfonyl)acetic acid (1.8 equiv) was added neat and dropwise over a period of 5 min. After another 48 h of stirring at RT, the crude reaction mixture was carefully quenched with water, and then diluted with a 1:1 (v/v) solution of ethyl acetate and hexanes. The organic layer was then separated and washed sequentially with saturated aq. NaHCO₃, water and brine, dried over MgSO₄, filtered, and the filtrate concentrated in vacuo. Purification of the crude product thus obtained by way of column chromatography (SiO₂, gradient elution: Hex→1:1 (v/v) Hex:EtOAc) afforded 2-(difluoromethoxy)-6-methoxy-3-nitropyridine as a yellow solid (75% yield).

Step 2: In a dried, round-bottom flask equipped with a magnetic stirrer was dissolved 2-(difluoromethoxy)-6-methoxy-3-nitropyridine (1 equiv) from the previous step in methanol (0.17 M). The resulting yellow solution was then deoxygenated via subsurface purging with nitrogen for 10 min before palladium (10% w/w over activated carbon, dry, 0.08 equiv) was added in one rapid portion. The resulting black suspension was then subsurface purged with hydrogen for 10 min before it was stirred under a static hydrogen atmosphere (maintained with a balloon) at RT for 90 min. The reaction was subsequently diluted with EtOAc and filtered through a bed of dichloromethane-wetted celite. The insolubles were washed further with EtOAc. Concentration of the filtrate thus obtained in vacuo afforded the title compound as a reddish, brown solid (>99% yield).

### Example 1: Preparation of trans-4-(3-(2-(difluoromethoxy)-6-methoxypyridin-3-yl)-1-(4-isopropylpyridin-3-yl)ureido)cyclohexane-1-carboxylic acid

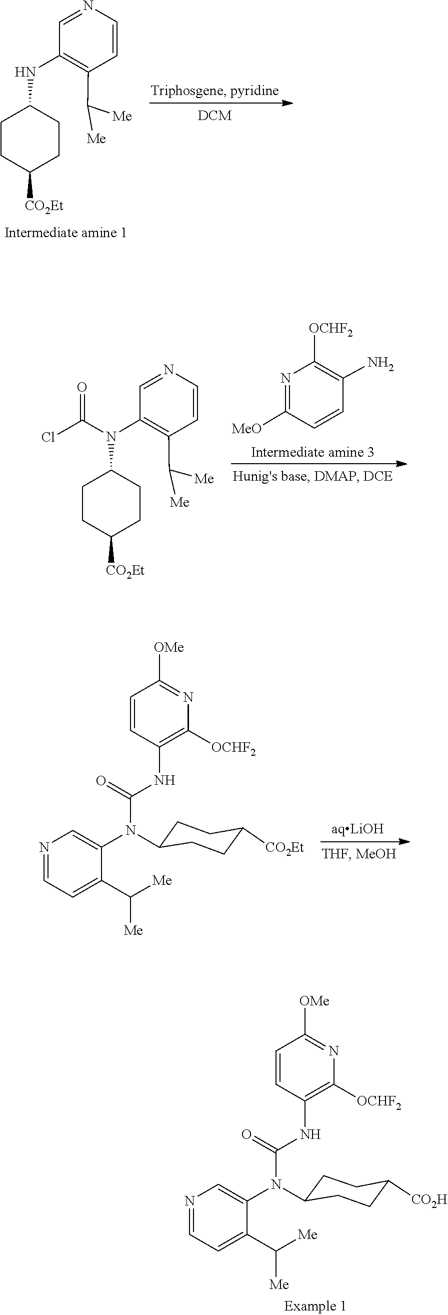 [SMILES: CCOC(=O)[C@H]1CC[C@H](Nc2cnccc2C(C)C)CC1 ; CCOC(=O)[C@H]1CC[C@H](N(C(=O)Cl)c2cnccc2C(C)C)CC1 ; COc1ccc(N)c(OC(F)F)n1 ; CCOC(=O)C1CCC(N(C(=O)Nc2ccc(OC)nc2OC(F)F)c2cnccc2C(C)C)CC1 ; COc1ccc(NC(=O)N(c2cnccc2C(C)C)C2CCC(C(=O)O)CC2)c(OC(F)F)n1]

Step 1: In a thick-walled glass reaction vessel equipped with a magnetic stirrer and a Teflon screwcap was combined Intermediate amine 1 (1 equiv) and pyridine (3 equiv) in anhydrous dichloromethane (0.45 M). To this was then added triphosgene (1 equiv) portionwise at RT over a period of 15 minutes. Following the completion of addition, the vessel was tightly sealed and heated at 60° C. for 1 h. The mixture was then cooled to 0° C. and carefully quenched with dropwise addition of methanol. The volatiles were then removed in vacuo and the crude product thus obtained was directly subjected to purification by way of column chromatography (SiO₂, gradient elution: DCM→10:1 (v/v) DCM:MeOH) to afford trans-ethyl 4-((chlorocarbonyl)(4-isopropylpyridin-3-yl)amino)cyclohexane-1-carboxylate as a yellow oil (71% yield).

Step 2: In a thick-walled glass reaction vessel equipped with a magnetic stirrer and a Teflon screwcap was combined trans-ethyl 4-((chlorocarbonyl)(4-isopropylpyridin-3-yl)amino)cyclohexane-1-carboxylate (1 equiv) from the previous step, Intermediate amine 3 (2.5 equiv), and DMAP (0.2 equiv) in anhydrous dichloroethane (0.28 M). To this was then added N,N-diisopropylethylamine (3 equiv) dropwise at RT over a period of 5 minutes. Following the completion of addition, the vessel was tightly sealed and heated at 110° C. for 16 h. The mixture was then cooled to RT, quenched with water, and extracted with EtOAc. The combined organic extracts were washed further with water and brine, dried over Na₂SO₄, filtered, and the filtrate concentrated in vacuo. Purification of the crude product thus obtained by way of column chromatography (SiO₂, gradient elution: 9:1 (v/v) Hex:EtOAc→EtOAc) afforded trans-ethyl 4-(3-(2-(difluoromethoxy)-6-methoxypyridin-3-yl)-1-(4-isopropylpyridin-3-yl)ureido)cyclohexane-1-carboxylate as a yellow oil (28% yield).

Step 3: In a round-bottom flask equipped with a magnetic stirrer was dissolved trans-ethyl 4-(3-(2-(difluoromethoxy)-6-methoxypyridin-3-yl)-1-(4-isopropylpyridin-3-yl)ureido)cyclohexane-1-carboxylate (1 equiv) from the previous step in a 1:1 (v/v) solution of THF and methanol (0.2 M). To this solution was then added lithium hydroxide (1 M solution in water, 5 equiv) and the resulting mixture was stirred at RT for 3 h. The reaction mixture was then carefully quenched with HCl (1 M solution in water, 5 equiv) and the volatiles were removed in vacuo. Purification of the crude product thus obtained by way of reverse phase column chromatography (C₁₈, gradient elution: 95:5 (v/v) H₂O:MeCN+10 mM NH₄HCO₃→5:95 (v/v) H₂O:MeCN+10 mM NH₄HCO₃) afforded the title compound as an off-white solid (81% yield). LCMS: m/z=479.6 [M+H]⁺; ¹H NMR (DMSO-d₆): d=10.23 (s, 1H), 8.54 (d, J=5.0 Hz, 1H), 8.37 (s, 1H), 7.80 (d, J=8.5 Hz, 1H), 7.60 (t, J=73.0 Hz, 1H), 7.52 (d, J=5.0 Hz, 1H), 6.91 (s, 1H), 6.61 (d, J=8.5 Hz, 1H), 4.21~4.16 (m, 1H), 3.81 (s, 3H), 3.14 (sept, J=6.5 Hz, 1H), 2.06~1.89 (m, 4H), 1.81~1.76 (m, 1H), 1.45~1.34 (m, 2H), 1.33~1.26 (m, 1H), 1.23 (d, J=6.5 Hz, 3H), 1.16 (d, J=6.5 Hz, 3H), 0.98~0.95 (m, 1H).

### Example 2: Preparation of trans-4-(3-(2-(difluoromethoxy)-6-methoxypyridin-3-yl)-1-(3-isopropylpyridin-2-yl)ureido)cyclohexane-1-carboxylic acid

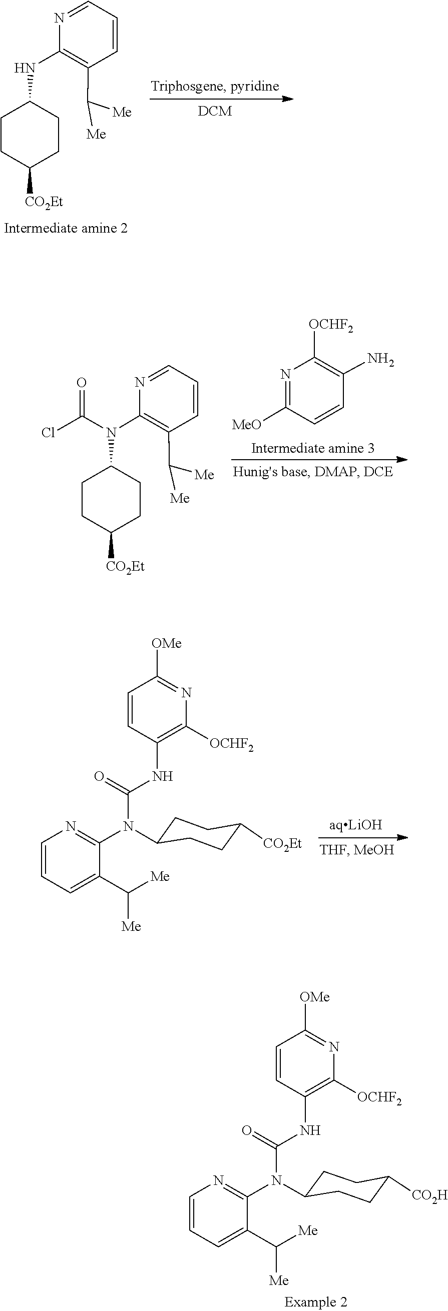 [SMILES: CCOC(=O)[C@H]1CC[C@H](Nc2ncccc2C(C)C)CC1 ; CCOC(=O)[C@H]1CC[C@H](N(C(=O)Cl)c2ncccc2C(C)C)CC1 ; COc1ccc(N)c(OC(F)F)n1 ; CCOC(=O)C1CCC(N(C(=O)Nc2ccc(OC)nc2OC(F)F)c2ncccc2C(C)C)CC1 ; COc1ccc(NC(=O)N(c2ncccc2C(C)C)C2CCC(C(=O)O)CC2)c(OC(F)F)n1]

Step 1: In a thick-walled glass reaction vessel equipped with a magnetic stirrer and a Teflon screwcap was combined Intermediate amine 2 (1 equiv) and pyridine (3 equiv) in anhydrous dichloromethane (0.43 M). To this was then added triphosgene (2 equiv) portionwise at RT over a period of 15 minutes. After the completion of addition, the resulting mixture was then stirred at RT for an additional 1 h. The mixture was then quenched with water and extracted with DCM. The combined organic extracts were washed further with water and brine, dried over Na₂SO₄, filtered, and the filtrate concentrated in vacuo. Purification of the crude product thus obtained by way of column chromatography (SiO₂, gradient elution: 9:1 (v/v) Hex:EtOAc→EtOAc) to afford trans-ethyl 4-((chlorocarbonyl)(3-isopropylpyridin-2-yl)amino)cyclohexane-1-carboxylate as a yellow oil (79% yield).

Step 2: In a thick-walled glass reaction vessel equipped with a magnetic stirrer and a Teflon screwcap was combined trans-ethyl 4-((chlorocarbonyl)(3-isopropylpyridin-2-yl)amino)cyclohexane-1-carboxylate (1 equiv) from the previous step, Intermediate amine 3 (2.5 equiv), and DMAP (0.2 equiv) in anhydrous dichloroethane (0.24 M). To this was then added pyridine (3 equiv) dropwise at RT over a period of 5 minutes. Following the completion of addition, the vessel was tightly sealed and heated at 110° C. for 2 h. The mixture was then cooled to RT, quenched with water, and extracted with DCM. The combined organic extracts were washed further with water and brine, dried over Na₂SO₄, filtered, and the filtrate concentrated in vacuo. Purification of the crude product thus obtained by way of column chromatography (SiO₂, gradient elution: 9:1 (v/v) Hex:EtOAc→EtOAc) afforded trans-ethyl 4-(3-(2-(difluoromethoxy)-6-methoxypyridin-3-yl)-1-(3-isopropylpyridin-2-yl)ureido)cyclohexane-1-carboxylate as a white solid (68% yield).

Step 3: In a round-bottom flask equipped with a magnetic stirrer was dissolved trans-ethyl 4-(3-(2-(difluoromethoxy)-6-methoxypyridin-3-yl)-1-(3-isopropylpyridin-2-yl)ureido)cyclohexane-1-carboxylate (1 equiv) from the previous step in a 1:1 (v/v) solution of THF and methanol (0.13 M). To this solution was then added lithium hydroxide (1 M solution in water, 3 equiv) and the resulting mixture was stirred at RT for 2 h. The reaction mixture was then carefully quenched with HCl (1 M solution in water, 3 equiv) and the volatiles were removed in vacuo. Purification of the crude product thus obtained by way of reverse phase column chromatography (C₁₈, gradient elution: 95:5 (v/v) H₂O:MeCN+10 mM NH₄HCO₃→5:95 (v/v) H₂O:MeCN+10 mM NH₄HCO₃) afforded the title compound as a white solid (63% yield). LCMS: m/z=479.4 [M+H]⁺; ¹H NMR (DMSO-d₆): d=11.87 (s, 1H), 8.42 (dd, J=4.8, 1.8 Hz, 1H), 7.94 (dd, J=7.8, 1.8 Hz, 1H), 7.85 (d, J=8.4 Hz, 1H), 7.60 (t, J=72.6 Hz, 1H), 7.44 (dd, J=7.8, 4.8 Hz, 1H), 6.86 (s, 1H), 6.61 (d, J=8.4 Hz, 1H), 4.13~4.07 (m, 1H), 3.80 (s, 3H), 3.15 (sept, J=6.0 Hz, 1H), 1.99~1.88 (m, 5H), 1.37~1.33 (m, 4H), 1.17 (d, J=6.8 Hz, 6H).

### 2. Biological Evaluations

### Example 3: In Vitro Functional Assay of Lysophosphatidic Acid Receptor 1 Activity

Primary compound plates were prepared in 100% DMSO (Sigma-Aldrich), secondary compound plates were prepared at 10× concentration in DMEM (Invitrogen), and tertiary compound plates were prepared at 3× concentration in assay buffer containing HBSS (no Ca⁺²/Mg⁺², Invitrogen) and 0.1% BSA (Sigma-Aldrich). Fluo-4 NW calcium assay dye (Invitrogen) was prepared as per manufacturer's recommendations in assay buffer. B103 cells stably expressing human LPA₁ (J. Chun lab, UCSD) were grown to confluency in DMEM media (Invitrogen) containing 10% FBS (ATCC), 10% Penicillin-Streptomycin (Sigma-Aldrich) and 50 μg Geneticin (Sigma-Aldrich), and detached with Accutase (Sigma-Aldrich) prior to assay. The freshly detached cells were resuspended in growth media and plated in black, clear-bottom 96-well plates (Costar) containing compound (i.e., secondary compound plate) at a density of 5×10⁴ cells/well. Once plated, cells were left at room temperature for 30 minutes and then transferred to a 37° C., 5% CO₂ incubator for 24 hours. After 24 hours, the growth media was then removed and freshly prepared Fluo-4 NW calcium assay dye was added to cells. Compounds (i.e., tertiary compound plates) were then added back to the dye/cells, returned to the incubator for 30 minutes, and then kept at room temperature for an additional 30 minutes. Finally, lysophosphatidic acid (18:1) at the EC₈₀ concentration was added and calcium flux measured using FlexStation 3 (Molecular Devices). Sigmoidal dose-response curves were generated by measuring luminescence over 45 sec and calculating the area under the curve. Dose response curves and IC₅₀ values were generated using Prism (GraphPad). Compounds were tested at a final concentration range of 100 pM to 10 μM in 0.1% DMSO. Results are shown in Table 1.

TABLE 1

| Example | LPA₁ IC₅₀ |
|---|---|
| 1 | B |
| 2 | B |

A = IC₅₀ of less than 10 nM;
B = IC₅₀ of less than 100 nM but greater or equal to 10 nM;
C = IC₅₀ of less than 1,000 nM but greater or equal to 100 nM;
D = IC₅₀ of greater or equal to 1,000 nM

TABLE 2
Additional biological data

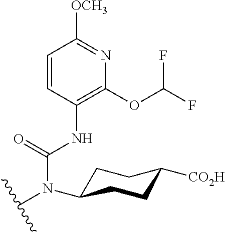

| | | | Example 3 |
|---|---|---|---|
| | | | (Comparator |
| | Example 1 | Example 2 | compound) |
| Chemical structure | 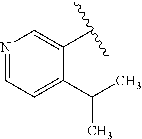 [SMILES: *c1cnccc1C(C)C] | 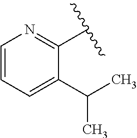 [SMILES: *c1ncccc1C(C)C] | 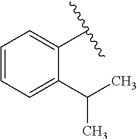 [SMILES: *c1ccccc1C(C)C] |
| Rat PK: Dosing PO @ 10 mpk | 0.05 | 0.15 | 0.47 |
| K_{p,uu} (unbound brain-to-plasma partitioning) | 0.29 | 2.1 | 5.2 |
| Brain concentration at 2 h (μM) | | | |

1.-6. (canceled)

7. A compound, or a pharmaceutically acceptable salt or solvate thereof, having the formula:

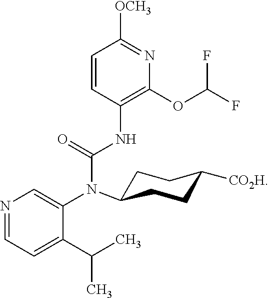

8.-14. (canceled)

15. A pharmaceutical composition comprising the compound of claim 7, or a pharmaceutically acceptable salt or solvate thereof, and a pharmaceutically acceptable excipient.

16. A method of treating a peripherally restricted LPAR1-associated disease in a subject in need thereof, said method comprising administering to the subject a therapeutically effective amount of the compound of claim 7, or a pharmaceutically acceptable salt or solvate thereof, wherein the peripherally restricted LPAR1-associated disease is Pulmonary fibrosis, renal fibrosis, liver fibrosis, nonalcoholic steatohepatitis, scleroderma, ocular fibrosis, or lung cancer.

17. The method of claim 16, wherein the peripherally restricted LPAR1-associated disease is pulmonary fibrosis, renal fibrosis, liver fibrosis, nonalcoholic steatohepatitis, scleroderma, or ocular fibrosis.

18. (canceled)

19. The method of claim 16, wherein the peripherally restricted LPAR1-associated disease is lung cancer.

20. (canceled)

21. A method of modulating LPAR1 activity in a subject, said method comprising administering to the subject the compound of claim 7, or a pharmaceutically acceptable salt or solvate thereof.
